SEQUENCE LISTING (4 sequences)

SEQ ID NO: 1 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 2 - protein, 1273 aa, Severe acute respiratory syndrome coronavirus 2
MFVFFVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 222 aa, Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 4 - protein, 75 aa, Severe acute respiratory syndrome coronavirus 2
MYSFVSEETGTLIVNSVLLFLAFVVFLLVTLAILTALRLCAYCCNIVNVSLVKPSFYVYSRVKNLNSSRVPDLLV

(19) United States
(12) Patent Application Publication
(10) Pub. No.: US 2023/0082940 A1
(43) Pub. Date: Mar. 16, 2023

(54) DEVICES AND METHODS FOR DETECTION OF SEVERE ACUTE RESPIRATORY SYNDROME CORONAVIRUS 2

(71) Applicants: INDIAN INSTITUTE OF TECHNOLOGY MADRAS (IIT MADRAS), Chennai (IN); RICOVR HEALTHCARE INC., St Princeton, NJ (US)

(72) Inventors: Raghavendra V.V. SAI, Chennai (IN); Himanshu BHATIA, St Princeton, NJ (US); Divagar MURUGAN, Chennai (IN)

(21) Appl. No.: 17/796,239

(22) PCT Filed: May 17, 2021
(86) PCT No.: PCT/IN2021/050474

§ 371 Date: Jul. 28, 2022

(30) Foreign Application Priority Data
May 15, 2020 (IN) 202041020644

(51) Int. Cl. G01N 21/552; G01N 21/77; G01N 33/569; G01N 33/543

(52) U.S. Cl. CPC G01N 21/554; G01N 21/7703; G01N 33/56983; G01N 33/54346; G01N 2021/258

(57) ABSTRACT

The invention discloses a biosensor device (100) to detect the presence of Severe Acute Respiratory Syndrome Coronavirus 2 (SARS-CoV-2) infection in a biological sample. The device includes an optical fiber probe (104) having a curved portion (104a) with a probe region (105) immobilized with bioreceptor molecules (201) configured to bind to the target molecule V indicative of the presence of the SARS-CoV-2. The probe has a light source (102) and a detector (106) on either end. The device works on the principle of plasmonic fiberoptic absorbance biosensing. Plasmonic gold nanoparticles (120) are used as either sensor substrate over the fiber or labels conjugated with a biorecognition molecule (211). The probe is exposed to a biological sample either directly for label-free detection, or after mixing with labels to realize a sandwich assay. The target biomolecules are detected by a proportional drop in the light intensity passing through the probe.

### CROSS-REFERENCES TO RELATED APPLICATIONS

This application claims priority to Indian patent application no. 202041020644 entitled “SYSTEMS AND METHODS FOR DETECTION OF SEVERE ACUTE RESPIRATORY SYNDROME CORONAVIRUS 2” filed on May 15, 2020.

### FIELD OF INVENTION

The present invention relates to biosensors and in particular, to a fiber optic biosensor device for detection of Severe Acute Respiratory Syndrome Coronavirus 2 (“SARS-CoV-2”) in a sample.

### DESCRIPTION OF THE RELATED ART

A biosensor is a biological analysis device for sensing a state and concentration of a target material such as biomolecular complexes, including oligonucleotides, antibody-antigen interactions, hormone-receptor interactions, and enzyme-substrate interactions. A biosensor typically includes a bioreceptor that senses and receives a target material, and a transducer that converts the sensed target material into a physically measurable signal. Optical biosensors are widely used in medicine for the purpose of analyzing bio-samples, such as blood, tissue cells, and the like, for their ease of operation and compatibility with other types of measurement techniques such as gravimetric and calorimetric techniques.

The coronavirus disease-2019 (COVID-19) outbreak, caused by SARS-CoV-2, already has had a major impact globally, in terms of both mortality and economics, since the first case reported from China in December 2019. The high infectivity and rapid spread of the virus have posed a serious threat across the globe, which can be witnessed as a steep rise in the mortality rate in the past four months. Mass screening for the identification of infected people (with or without symptoms) and their isolation and appropriate treatment have shown positive progress to break the chain of community transmission. Currently, the reverse transcription-polymerase chain reaction (RT-PCR) technique is widely used to detect SARS-CoV-2, which usually takes a few hours for the analysis. However, its wide-scale deployment in resource-constrained settings is limited as it needs expensive equipment and trained personnel for performing complicated sample preparation steps and use of specialized equipment. Considering a large population with suspected or confirmed SARS-CoV-2 infection, there is an urgent need for a rapid diagnostic tool that does not rely on trained personnel or expensive equipment.

While antibody-based diagnostic assays for SARS-CoV-2 provide rapid analysis, they are based on the serological determination of the neutralizing antibodies produced in the host as a defense response against SARS-CoV-2. However, delayed immune response along with large variations in the serum IgM/IgG antibody level in the infected persons may pose the risk of false-positive/negative results. Since SARS-CoV-2 could re-emerge and cause another epidemic at any time, development of rapid detection assays that can detect the presence of SARS-CoV in early stages accurately is of vital importance.

The US Publication US20110207237A1 describes a biosensor having an optical fiber having at least one curved portion configured to enhance penetration of evanescent waves. The Chinese Publication CN110763659A describes a biosensor with surface plasmon resonance (SPR) effect to enhance a surface field of the sensor. A localized surface plasmon resonance (LSPR) optic probe with gold nanoparticles (GNPs) for clinical research and for protein detection as described in “Plasma Enhanced Label-Free Immunoassay for Alpha-Fetoprotein Based on a U-Bend Fiber-Optic LSPR Biosensor” Liang et al. (2015). Gold-coated U-shaped plastic optical fiber (POF) biosensor for E. coli bacteria detection is described in “Surface Plasmon Resonance And Bending Loss-Based U-Shaped Plastic Optical Fiber Biosensors” Arcas et al. (2018). U-bent probe with simple optoelectronic instrumentation having an LED and a fiber optic spectrometer for several bio-sensing applications as described in “Evanescent Wave Absorbance Based U-Bent Fiber Probe for Immuno-biosensor With Gold Nanoparticle Labels”, Ramakrishna and Sai (2015).

The invention proposes devices and methods to address some of the drawbacks discussed here.

### SUMMARY OF THE INVENTION

The invention discloses devices and methods for detection of Severe Acute Respiratory Syndrome Coronavirus 2 (SARS-CoV-2) or fragment thereof in a biological sample. The invention in various embodiments discloses a biosensor device 100 for detecting Severe Acute Respiratory Syndrome Coronavirus 2 (SARS-CoV-2) or fragment thereof in a biological sample. The biosensor device comprises an optical fiber 104 comprising at least one curved portion 104a, and a probe region 105, the probe region comprising a plurality of immobilized bioreceptor molecules 201 configured to bind to target biomolecules associated with SARS CoV-2 infection in the sample. A light source 102 is located proximal to one end of the optical fiber 104, and a detector 106 is located proximal to another end of the optical fiber 104, wherein the detector is configured to sense a change in an optical property of light that traverses through the optical fiber when the probe region 105 is contacted with a biological sample including the target biomolecules.

In some embodiments the probe region 105 comprises a coating 120 of gold or silver nanoparticles for immobilizing the bioreceptor molecules. In some embodiments the plurality of bioreceptor molecules comprise an antibody configured to bind to an antigen of the SARS-CoV-2. In some embodiments the antigen is one or more of N (nucleocapsid (N) glycoprotein) according to SEQ. ID. No. 1, S (Spike Glycoprotein) according to SEQ. ID. No. 2, M (Membrane protein) according to SEQ. ID. No. 3, or E (Envelop small protein) according to SEQ. ID. No. 4, of the SARS-CoV-2.

In various embodiments the biological sample may comprise saliva, nasopharyngeal or oropharyngeal swab collected from a subject. In various embodiments, the bioreceptor molecules are anti-SARS CoV-2 polyclonal or monoclonal antibody against the antigen. In various embodiments the optical fiber is made of a transparent material selected from silica, quartz, polymethyl methacrylate, polystyrene, ceramic glass, or chalcogenide glass. In some embodiments the nanoparticles 120 are spherical or elliptical gold nanoparticles of size 15-60 nm.

In various embodiments, a labelled assay method for detecting Severe Acute Respiratory Syndrome Coronavirus 2 (SARS-CoV-2) in a sample, is disclosed. The method comprises the steps of providing (401) an optical probe biosensor device having a U-bent probe region, and immobilizing (403) a bioreceptor configured to bind to target biomolecules associated with SARS CoV-2 to the probe region. Then the biological sample is mixed (405) with gold nanoparticle labels conjugated with a biorecognition molecule specific to a SARS-CoV-2 antigen and incubated to allow formation of an AuNP-antibody-antigen complex. Exposing (407) the probe region to the sample-label mixture then allows binding of the target biomolecules and formation of a sandwich immunocomplex with gold nanoparticle labels. In the next step, light is passed through the optical fiber and detecting (409) a change in intensity of the light passing through the optical probe biosensor as a function of the amount of target biomolecules associated with SARS CoV-2 forming the immunocomplex.

In some embodiments the method comprises functionalizing (402) the U-bent sensor probe surface with —OH or —CHO groups prior to immobilization of the bioreceptor. In various embodiments, the bioreceptor molecule or the biorecognition molecule comprises an antibody configured to bind to an antigen of the SARS-CoV-2, selected from one or more of N (nucleocapsid (N) glycoprotein) according to SEQ. ID. No. 1, S (Spike Glycoprotein), according to SEQ. ID. No. 2, M (Membrane protein) according to SEQ. ID. No. 3, E (Envelop small protein) according to SEQ. ID. No. 4, or (HE) (hemagglutinin-esterase) protein of the SARS-CoV-2.

In various embodiments, a label-free assay method for detecting Severe Acute Respiratory Syndrome Coronavirus 2 (SARS-CoV-2) in a sample, is disclosed. The method comprises providing (501) an optical probe biosensor device having a U-bent probe region and providing (503) a coating of gold nanoparticles on the U-bent probe region. Then a bioreceptor configured to bind to target biomolecules associated with SARS CoV-2 is immobilized (505) to the nanoparticle-coated probe region. Then, exposing (507) a biological sample to the probe region causees the target biomolecules to bind to the bioreceptor and form an immunocomplex. In the next step, light is passed through the optical fiber and (509) a change in intensity of the light passing through the optical biosensor probe is detected as a function of the amount of target biomolecules associated with SARS CoV-2 forming the immunocomplex.

In some embodiments, the label-free assay method may comprise functionalizing (502) the U-bent sensor probe surface with —SH or —NH₂ groups prior to coating with gold nanoparticles and immobilizing the bioreceptors. In various embodiments, the bioreceptor molecule comprises an antibody configured to bind to an antigen of the SARS-CoV-2, selected from one or more of N (nucleocapsid (N) glycoprotein) according to SEQ. ID. No. 1, S (Spike Glycoprotein), according to SEQ. ID. No. 2, M (Membrane protein) according to SEQ. ID. No. 3, or E (Envelop small protein) according to SEQ. ID. No. 4, of the SARS-CoV-2.

This and other aspects are disclosed herein.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 illustrates an example biosensor device.

FIG. 2A is a schematic of the biosensor device set up for labelled assay.

FIG. 2B is a schematic of label-free assay using the biosensor device.

FIG. 2C illustrates labelled assay method.

FIG. 2D illustrates label-free assay method.

FIG. 3 illustrates components of a system for sensing using the biosensor device.

FIG. 4 shows a schematic representation of plasmonic fiber optic absorbance biosensor (P-FAB) strategy for the detection of SARS-CoV-2 N-protein and the LED-PD based experimental set-up used for the optical absorbance measurements.

FIG. 5A shows the temporal response from the U-bent POF probes obtained by physisorption.

FIG. 5B shows the temporal response from the U-bent POF probes obtained by HMDA based covalent immobilization of antibodies.

FIG. 5C shows dose-response curve of POF sensor probes with capture antibody (Anti-SARS N-protein IgG1) immobilized by means of physisorption, Acid-treatment, HMDA, and EDC/NHS based covalent binding methodology.

FIG. 6 shows absorbance response obtained from POF and GOF sensor probes chemisorbed with anti-SARS-CoV-2 -N-protein IgG1 due to the binding of AuNP-SARS-CoV-2 N-protein complex of varying concentration (ng/mL).

FIG. 7A shows representative temporal absorbance response.

FIG. 7B shows dose-response curves obtained from GOF sensor probes chemisorbed with anti-SARS-CoV-2-N-protein IgG.

FIG. 7C shows TEM images of prepared anisotropic AuNPs.

FIG. 8 shows dose response curve obtained using covalently immobilized capture antibody functionalized sensor probes for various dilutions of SARS-CoV-2 N-protein sample from Indian Council for Medical Research (ICMR).

FIG. 9A shows temporal absorbance for different dilutions of saliva sample.

FIG. 9B illustrates the maximum absorbance response obtained from GOF sensor probes using P-FAB strategy with different dilutions of saliva.

Referring to the figures, like numbers indicate like parts throughout the views.

### DETAILED DESCRIPTION OF THE EMBODIMENTS

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to.” When used in this document, the term “exemplary” is intended to mean “by way of example” and is not intended to indicate that a particular exemplary item is preferred or required.

When used in this document, terms such as “top” and “bottom,” “upper” and “lower”, “inner” and “outer”, or “front” and “rear,” are not intended to have absolute orientations but are instead intended to describe relative positions of various components with respect to each other. For example, a first component may be an “upper” component and a second component may be a “lower” component when a device of which the components are a part is oriented in a first direction. The relative orientations of the components may be reversed, or the components may be on the same plane, if the orientation of the structure that contains the components is changed. The claims are intended to include all orientations of a device containing such components.

The term “antibody” (Ab) as used herein includes monoclonal antibodies, polyclonal antibodies, multispecific antibodies (for example, bispecific antibodies and polyreactive antibodies), and antibody fragments. Thus, the term “antibody” as used in any context within this specification is meant to include, but not be limited to, any specific binding member, immunoglobulin class and/or isotype (e.g., IgG1, IgG2, IgG3, IgG4, IgM, IgA, IgD, IgE); and biologically relevant fragment or specific binding member thereof, including but not limited to Fab, F(ab′)2, Fv, and scFv (single chain or related entity). It is understood in the art that an antibody is a glycoprotein comprising at least two heavy (H) chains and two light (L) chains inter-connected by disulfide bonds, or an antigen binding portion thereof. A heavy chain is comprised of a heavy chain variable region (VH) and a heavy chain constant region (CH1, CH2 and CH3). A light chain is comprised of a light chain variable region (VL) and a light chain constant region (CL). The variable regions of both the heavy and light chains comprise framework regions (FWR) and complementarity determining regions (CDR). The four FWR regions are relatively conserved while CDR regions (CDR1, CDR2 and CDR3) represent hypervariable regions and are arranged from NH2 terminus to the COOH terminus as follows: FWR1, CDR1, FWR2, CDR2, FWR3, CDR3, FWR4. The variable regions of the heavy and light chains contain a binding domain that interacts with an antigen while, depending of the isotype, the constant region(s) may mediate the binding of the immunoglobulin to host tissues or factors. The term “monoclonal antibody” as used herein refers to an antibody obtained from a population of substantially homogeneous antibodies, i.e., the individual antibodies comprising the population are identical except for possible naturally occurring mutations that may be present in minor amounts. The term “polyclonal antibody” refers to preparations that include different antibodies directed against different determinants (“epitopes”).

Also included in the definition of “antibody” as used herein are chimeric antibodies, humanized antibodies, and recombinant antibodies, human antibodies generated from a transgenic non-human animal, as well as antibodies selected from libraries using enrichment technologies available to the artisan.

As used herein, the term “biological sample” or “sample” encompasses a variety of sample types, including blood and other liquid samples of biological origin (e.g., blood, plasma, serum, gastrointestinal secretions, homogenates of tissues or tumors, synovial fluid, feces, saliva, nasopharyngeal or oropharyngeal swab, sputum, cyst fluid, amniotic fluid, cerebrospinal fluid, peritoneal fluid, lung lavage fluid, semen, lymphatic fluid, tears, and prostatic fluid), solid tissue samples such as a biopsy specimen or tissue cultures, or cells derived therefrom and the progeny thereof. The term also includes samples that have been manipulated in any way after their procurement, such as by treatment with reagents, solubilization, or enrichment for certain components, such as proteins or polynucleotides. The term encompasses various kinds of clinical samples obtained from any species, and also includes cells in culture, cell supernatants, and cell lysates. Detected target molecules present in a biological sample indicative of SARS-CoV in a subject may include, e.g., hormones, different pathogens, proteins, antibodies or other chemical or biological substances. Bodily fluid liquid samples of biological origin may include, e.g., blood, urine, saliva, nasopharyngeal or oropharyngeal swab, tears, ejaculate, odor or other body fluids. In some embodiments, the sample used may be selected from saliva, nasopharyngeal swab or oropharyngeal swab.

As used herein, the term “biomolecules” is intended to be a generic term, which includes for example (but not limited to) SARS-CoV itself or proteins such as antibodies or cytokines, peptides, nucleic acids, lipid molecules, polysaccharides, etc. indicative of SARS-CoV infection.

SARS-CoV is a single-stranded, positive-sense RNA virus, phylogenetically related to coronaviruses from group 2 despite the fact that it does not encode a hemagglutinin-esterase protein (Snijder E. J., Bredenbeek P. J., Dobbe J. C. Unique and conserved features of genome and proteome of SARS-coronavirus, an early split-off from the coronavirus group 2 lineage. J. Mol. Biol. 2003;331(5):991-1004.). The genome is packaged together with the nucleocapsid (N) protein, at least five membrane proteins (M, E, 3a, 7a, and 7b) and the spike (S) protein). Nucleocapsid (N) glycoproteins have been reported to be found in abundance during early stages of infection with SARS-CoV-2.

One aspect of the present disclosure provides a SARS-CoV-2 detection biosensor device 100. Specifically, the SARS-CoV-2 detection biosensor device may include a fiber optic probe including a functionalized region on which bioreceptor molecules are immobilized. In some embodiments, gold (AuNPs) (or other metallic) nanoparticles are used as a substrate in the functionalized region for immobilizing the bioreceptor molecules. The bioreceptors function as a capture agents immobilized to the substrate and are configured to bind to SARS-CoV-2 related target biomolecules in a biological sample being analyzed (directly and/or indirectly).

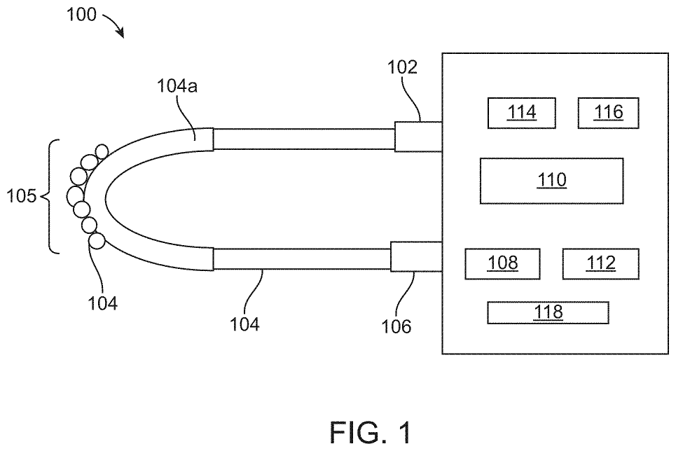

In various embodiments is disclosed in FIG. 1, a schematic diagram of a biosensor device 100 for detection of SARS-CoV-2 related target biomolecules in a sample is shown. As shown in FIG. 1, the biosensor device 100 is a single-channel fiber optic device that allows analysis of one sample at a time. However, the disclosure is not so limiting and multiple samples may be simultaneously analyzed without deviating from the principles described.

In various embodiments the biosensor device 100 includes a light source 102 located proximate to one end of an optical fiber 104 for illuminating the optical fiber 104 at a predetermined wavelength. The optical fiber 104 may be bent to form a U-shape such that light from the light source 102 may traverse the U-bent region (104a) of the optical fiber 104 before being received by a detector 106 located proximate to another end of the optical fiber 104. A portion of the optical fiber 104 may include a probe region 105 that is functionalized or immobilized with a plurality of immobilized bioreceptor molecules 201. The bioreceptor molecules are configured to bind to target biomolecules associated with SARS CoV-2 infection in the subject, where the binding causes a change in the properties of light traversing the optical fiber 104 that may be detected by the detector 106. In some embodiments, at least a part of the U-bent region 104a may form the probe region 105. In some embodiments, an uncladded portion of the U-bent region 104a (e.g., the tip) may form the probe region 105. Optionally, at least a part of the straight arm 104b forms the probe region 105.

In various embodiments, the plurality of bioreceptor molecules comprise an antibody capable of binding to an antigen of the SARS-CoV-2. In some embodiments the antigen may be one or more of N (nucleocapsid (N) glycoprotein) SEQ. ID. NO. 1 according to NCBI accession no. QII57164, S (Spike Glycoprotein) SEQ. ID. NO. 2 according to NCBI accession no. QIU81910, M (Membrane protein), SEQ. ID. NO. 3 according to NCBI accession no. QII57163, or E (Envelop small protein) SEQ. ID. NO. 4 according to NCBI accession no. No: QIH45055 protein of the SARS-CoV-2.

At the U-bent region, light interacts with biomolecules bound to the bioreceptor and traverses through the second arm of the optical fiber towards the detector end. For example, an interaction with a target biomolecule may lead to modulation in the effective refractive index (RI) and/or evanescent wave absorbance of light waves traversing through the functionalized portion as a function of target biomolecule type, concentration, and/or other properties. The interaction signal may be amplified by coating the probe region 105 with gold or silver nanoparticles 120 (or other metallic film) that may, for example, lead to greater loss in the light and may be measured usually as an increase in the absorbance value (or decrease in the intensity count). Immobilization of bioreceptors and immunocomplex formation during biomolecule binding on the surface of gold nanoparticles leads to increase in effective refractive index of the microenvironment causing a change in properties of light traversing through the optical fiber. Further, the application of U-bent fiber facilitates the placement of light source 102 and detector 106 and eliminates the use of additional optical components including beam splitters. In some embodiments the gold or silver nanoparticles 120 may be of size 15-60 nm.

In various embodiments the light source 102 may include, for example, one or more light emitting diodes (LEDs) configured to emit light in the visible, infrared (IR), and/or ultraviolet (UV) wavelengths. In some embodiments, a green, red, yellow, or another color LED may be used. Light emitted by the light source 102 may, optionally, be controlled using a feedback circuit based on signal or information received by the detector 106. For example, current driven to the light source may be maintained at the desired level.

In various embodiments the detector 106 may correspondingly operate in the visible, infrared (IR), and/or ultraviolet (UV) wavelengths. Examples of the detector 106 may include, without limitation, a spectrometer (spectrophotometer, spectrograph, spectroscope, or other suitable device), an optical detector sensitive at a particular localized surface plasmon resonance (LSPR) frequency, photodiode, phototransistor, or the like.

In various embodiments the probe region 105 formed by coating a portion of the optical fiber (i.e., functionalized) with suitable bioreceptor molecules 101 or 201 (directly and/or bioreceptor conjugated with the metallic nanoparticle) will now be described in detail below. The coating facilitates the binding of the target biomolecules to the surface of the probe region. The bioreceptor molecules may include a protein, an antibody, an antigen, an enzyme, a nucleic acid, a microorganism, or the like; and metallic nanoparticles may include gold/silver.

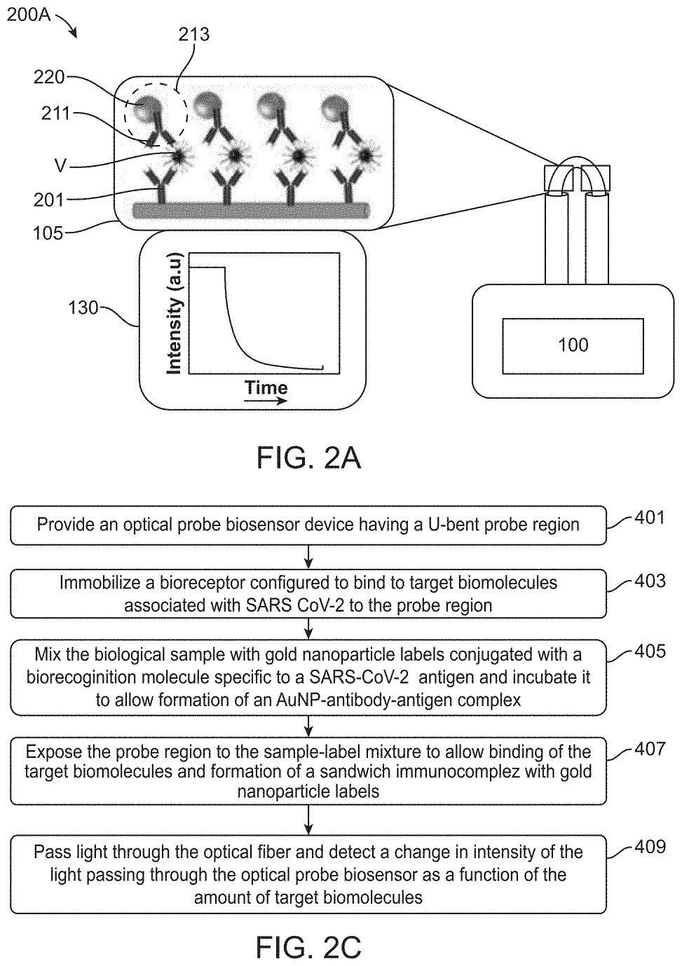

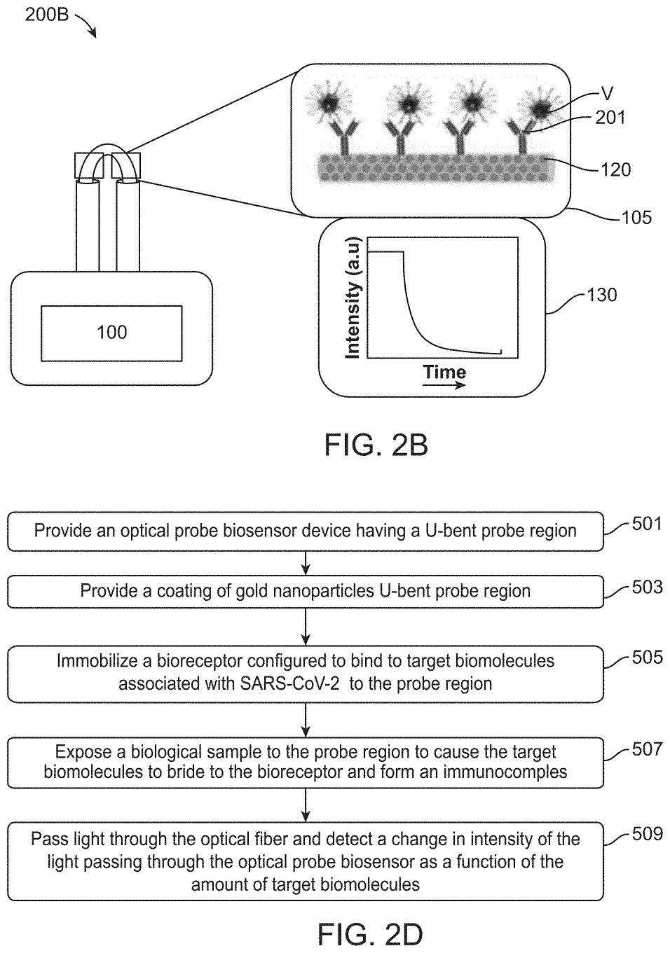

In some embodiments, the probe region 105 maybe the uncladded exposed fiber core with metallic nanoparticle coating on the uncladded exposed fiber core. Typically, the complete probe region or a portion of the probe region comes in contact with the target biomolecules. For SARS Cov-2 related biomolecules, the probe region 105 may be coated with a specific bioreceptor depending on the biomolecule being targeted for detection. Specifically, bioreceptors are substances used as specific indicators for detecting the presence of specific biomolecules. In various embodiments, the detection of SARS-CoV-2 specific biomolecules using bioreceptors may be divided in two categories: (a) the labeled techniques and (b) the label-free techniques, as further disclosed with reference to FIGS. 2A and 2B.

In one embodiment, a method 200A of detecting a virus fragment V via a labeled assay is shown in FIGS. 2A and 2C. The method (FIG. 2C) may include in step 401, providing an optical probe biosensor device 100 having a U-bent probe region, as already discussed with reference to FIG. 1. The next step 403 involves immobilizing a bioreceptor configured to bind to target biomolecules associated with SARS CoV-2 to the nanoparticle-coated probe region. As illustrated in FIG. 2A, the probe region 105 may be uncladded portion of the optical fiber. In step 403, the probe region 105 may be coated with a plurality of bioreceptor molecules 201 configured to detect a SARS-CoV-2 specific target biomolecule V. The sample having the target biomolecule V may then be mixed in step 405 with gold or silver nanoparticles 220 conjugated with biorecognition molecules 211, forming conjugate 213. In various embodiments, the conjugated biorecognition molecules 211 may either be the same as bioreceptor molecule 201 or may be a different antibody configured to capture the target biomolecule. In the next step 407, the probe region is exposed to the sample-label mixture to allow binding of the target biomolecules and formation of a sandwich immunocomplex with gold nanoparticle labels. Light is then passed through the fiber and a change in intensity of the light passing through the optical probe biosensor is detected 409 as a function of the amount of target biomolecules associated with SARS CoV-2 forming the immunocomplex.

In various embodiments, a label-free assay method 200B is illustrated with reference to FIGS. 2B and 2D. The method (FIG. 2D) involves providing in step 501 an optical probe biosensor device having a U-bent probe region as already illustrated with reference to FIG. 1. Then, a coating of gold or silver nanoparticles is given in step 503 on the U-bent probe region. Thereafter, a bioreceptor configured to bind to target biomolecules associated with SARS CoV-2 is immobilized in step 505 to the nanoparticle-coated probe region. Then the biological sample is exposed (507) to the probe region to cause the bioreceptor to bind to the target biomolecules and form an immunocomplex. By passing light through the optical fiber, a change in intensity of the light passing through the optical probe biosensor is detected in step 509 as a function of the amount of target biomolecules associated with SARS CoV-2 forming the immunocomplex.

In various embodiments of the methods 200A and 200B, the methods may further involve functionalizing the probe surface 105. The fiber probe surface in step 402 of the labelled assay method may be functionalized with —OH or —CHO groups prior to step 402 of immobilizing the bioreceptor.

In some embodiments, the fiber probe surface in step 502 of the label-free assay method may be functionalized by forming —SH or —NH₂ groups prior to step 503 of coating with gold nanoparticles and immobilizing (step 505) the bioreceptor.

In some embodiments, the fiber may be a polymeric optical fiber and the functionalizing may be done with —OH groups by treating sequentially with 1 M H₂SO₄ for 1 hr followed by incubating in methanol:HCl (1:1) for 1 hr at room temperature (RT) to generate hydroxyl (—OH) groups on the surface. Further treatment with hexamethyl diamine is done to obtain NH₂ groups.

In some embodiments the fiber may be a silica fiber and the probe surface may be amino- or mercapto-silanized for functionalization with —NH₂ or —SH groups respectively. The fibers are first piranha treated to produce hydroxyl groups on the surface. For amino-silanization the fiber probes are dipped in a 1% solution of APTMS in a 5:2 (v/v) mixture of ethanol and acetic acid (5 min), followed by hexamethyl diamine (HMDA) to obtain NH₂ groups.

Finally, the fiber probes are washed thrice in ethanol, sonicated (15 min) and dried (100° C., 1 h). Then, the probes are coated with gold nanoparticles (AuNP) by incubating them in AuNP solution until they show optical absorbance of up to 2.0 units.

The bioreceptors are immobilized on the AuNP surface over the probes with either directly or through cross linkers such as cystamine followed by glutaraldehyde. Then, the silanized sensor probes are dipped into 1% glutaraldehyde (500 μL, 30 min, RT) to establish aldehyde groups —CHO.

In various embodiments of the methods 200A and 200B, the bioreceptor or the biorecognition molecule may be an antibody capable of binding to an antigen of the SARS-CoV-2. In some embodiments, the antigen may be selected from one or more of N (nucleocapsid (N) glycoprotein) SEQ. ID. NO. 1 according to NCBI accession no. QII57164, S (Spike Glycoprotein) SEQ. ID. NO. 2 according to NCBI accession no. QIU81910, M (Membrane protein), SEQ. ID. NO. 3 according to NCBI accession no. QII57163, or E (Envelop small protein) SEQ. ID. NO. 4 according to NCBI accession no. No: QIH45055 protein of the SARS-CoV-2. The presence and amount of biomolecules present are then detected by passing light through the probe and measuring absorption of evanescent waves caused by localized surface plasmon resonance (LSPR). The method of detection of SARS-CoV-2 specific target biomolecule V using the device 100 is further illustrated.

The bioreceptor included in the SARS-CoV-2 detection sensor may refer to an agent configured to sense, immobilize or capture target biological molecules (e.g., N-proteins of SARS-CoV-2) to be measured and detected in a sample. In the present invention, the target biological molecules may include protein molecules associated with SARS-CoV-2. The bioreceptor may employ, for example and without limitation, anti-N protein monoclonal antibodies as a capture agent configured to bind to N-protein molecules associated with SARS-CoV-2 in a biological sample in order to allow N-protein molecules associated with SARS-CoV-2 to be immobilized to the probe region 105. Any signal indicative of N-protein molecules bound to the bioreceptor may be indicative of a SARS-CoV-2 infection in a subject from which the biological sample is collected. Similarly, antibodies configured to bind to other components of the SARS-CoV-2 may be used as bioreceptors. In some embodiments, the amount of bioreceptors immobilized on the probe region may be optimized based on a desired sensitivity of the sensors device (i.e., an amount required to detect a threshold concentration of the target biomolecule).

The term “optical fiber or fiber optic” as used in this document refers to a waveguide that can transfer light from one end to other by internal reflection of light within the fiber. An optical fiber may be cladded or uncladded. Cladded optical fibers generally have a structure from the center to the periphery including core, cladding, and buffer. An uncladded optical fiber, lacking cladding, generally has an exposed core. The core may be made of any transparent material such as silica (glass) or a polymer that transmits light. In cladded fiber optics, the core is typically surrounded by, but not limited to, a thin plastic or silica cladding that helps transmit light through the optical fiber with minimum loss. The cladding may be covered with a tough resin buffer layer. Both the core and the cladding may be made of dielectric materials such as, but not limited to, doped silica glass and polymers. To confine the optical signal in the core, the refractive index of the core is typically greater than that of the cladding.

In various embodiments the refractive index for the cladding of the optical fiber 104 may be about 1.390-1.460, about 1.392-1.458, about 1.394-1.456, about 1.396-1.454, about 1.398-1.450, about 1.40, about 1.398, about 1.47, or the like. Example values of the refractive index for the core of the optical fiber 104 is greater than that of the cladding such as, for example, about 1.448-1.50, about 1.458-1.49, about 1.47-1.49, about 1.46, about 1.47, about 1.458, about 1.40, about 1.48, about 1.49, about 1.50 or the like. The boundary between the core and cladding may either be abrupt, in step-index fiber, or gradual, in graded-index fiber. In certain embodiments, the radius of the optical fiber (core plus cladding) may be from about 0.1 mm to about 2 mm, about 0.2 mm to about 1.9 mm, about 0.22 mm to about 0.65 mm, about 0.3 mm to about 1.8 mm, about 0.4 mm to about 1.7 mm, about 0.5 mm to about 1.6 mm, about 0.6 mm to about 1.5 mm, about 0.7 mm to about 1.4 mm, about 0.8 mm to about 1.3 mm, about 0.9 mm to about 1.2 mm, about 0.2 mm to about 0.5 mm, about 0.3 mm to about 0.4 mm, about 0.2 mm, about 0.3 mm, about 0.4 mm, about 0.5 mm, or the like. The thickness of the cladding may be about 10 μm to about 20 μm, bout 12 μm to about 18 μm, bout 14 μm to about 16 μm, or thief like. In some embodiments, the optical fiber includes a core and a cladding, however the cladding is partially removed. That is, the cladding is removed over a portion of the optical fiber, the remaining fiber maintaining its cladding.

The term “evanescent waves” in the context of the optics used in this disclosure refer to waves that are formed at the core/cladding interface as the light passing through the fiber core undergoes total internal reflection. In all optical fibers, light propagates by means of total internal reflection, wherein the propagating light is launched into waveguide at angles such that upon reaching the cladding-core interface, the energy is reflected and remains in the core of the fiber. For light reflecting at angles near the critical angle, a significant portion of the power extends into the cladding or medium which surrounds the core. This phenomenon, known as the evanescent wave (EW), extends only to a short distance from the interface, with power dropping exponentially within a distance of λ/10 (typically less than 50 nm) from the core/cladding interface. This is called an evanescent field or evanescent wave (EW). Cladding material (either polymer or silica) is generally removed in order to access these evanescent waves. The cladding material may be removed, for example, by using a sharp surgical blade, a file, sand paper, or any other abrasive tool.

When a molecule is bound (e.g., immobilized by functionalization), to an uncladded portion of the optical fiber, it may absorb the evanescent wave, and the measured absorption spectra may be used to detect presence, concentration and/or other properties of the molecule, as explained in more detail below. This is called evanescent wave absorbance phenomenon.

Evanescent wave based absorbance sensitivity of bare (unclad, uncoated surface of fiber core) fiber optic probes may be increased by modifying the probe geometry. Different fiber optic probe designs including straight, U-bent, tapered tip, and biconical tapers may be employed in development of absorbance based bio/chemical sensors. Some embodiments relate to U-bent probes having one or more of (and not limited to) good sensitivity, compactness, ease in fabrication, and possibly higher compatibility with instrument configurations. Evanescent fields around U-bent probes are stronger than in a straight probe.

Although the length of biosensor probe and the resolution of a biosensor are directly proportional, the length of the probe cannot be increased beyond a certain limit as the sample analyte volume required also increases. The biosensors of the embodiments herein, however maybe length independent because of the U-bent, have increased sensitivity and could be configured for use with low sample volume of 100 microliters or less. However, if a larger or wider flowcell is used, a larger sample volume may be accommodated. Indeed, the biosensor may be used without a flow cell—the biosensor can work in any body of analyte, for example, a beaker, cup, glass, pond or river. The embodiments herein satisfy the need for a biosensor with increased sensitivity and/or resolution irrespective of the length.

The embodiments relate to a U-bend shaped fiber optic based biosensor having improved sensitivity, which depends on the bend diameter and the length of the uncladded optical fiber after the bent region for a given radius of the fiber core. As shown in FIG. 1, the bend in fiber forces the light travelling along the center (optical axis) of fiber to come to periphery and increases penetration of waves into samples. Among the embodiments herein, the U-bent shaped fiber optic includes a straight optical fiber (e.g., having 0.2 mm of core diameter) bent in to a substantially U shape, typically bent 180 degrees. The bend, however, may be less than 180 degrees or greater than 180 degrees. In some embodiments, the bend may be about 170 degrees to about 190 degrees, about 172 degrees to about 188 degrees, about 174 degrees to about 186 degrees, about 176 degrees to about 184 degrees, about 178 degrees to about 182 degrees, or the like. In an embodiment, the bend may vary from 90 degrees to 270 degrees. In another embodiment, the bend may vary from 1 degree to 359 degrees. In another embodiment, the “U” shape has flat bottom. That is, the U is made of three straight line segments. In still another embodiment, the optical fiber has substantially a V shape.

In certain embodiments, surface plasmon resonance (SPR) may be used to further increase sensitivity and/or accuracy of detection by coating the surface of the optical fiber under cladding with a thin film of metal such as silver or gold nanoparticles that act as substrate for immobilization of the bioreceptor. A surface plasmon is an oscillation of free electrons that propagates along the surface of a conductor. The phenomenon of SPR occurs under total internal reflection conditions at the boundary between substances of different refractive indices, such as glass and water solutions. When an incident light beam is reflected internally within the first medium, its electromagnetic field produces an evanescent wave that crosses a short distance (in the order of nanometers) beyond the interface with the second medium. If a thin metal film is inserted at the interface between the two media, surface plasmon resonance occurs when the free electron clouds in the metal layer (the plasmons) absorb energy from the evanescent wave and cause a measurable drop in the intensity of the reflected light at a particular angle of incidence that depends on the refractive index of the second medium. Surface plasmon resonance reflectivity measurements, as discussed in more detail below, may be used to detect molecular adsorption, such as by SARS-CoV-2 related biomolecules in a sample.

Typically, the conductor used for SPR spectrometry is a thin film of metal such as silver or gold; however, surface plasmons have also been excited on semiconductors, noble-metal containing nanoparticle includes one or more of rhodium, iridium, palladium, silver, osmium, iridium, platinum, gold or combinations thereof, or any material which exhibits surface plasmon resonance (e.g., copper and aluminum). The conventional method of exciting surface plasmons is to couple the transverse-magnetic (TM) polarized energy contained in an evanescent field to the plasmon mode on a metal film. The amount of coupling, and thus the intensity of the plasmon, is determined by the incident angle of the light beam and is directly affected by the refractive indices of the materials on both sides of the metal film. By including the sample material to be measured as a layer on one side of the metallic film, changes in the refractive index of the sample material may be monitored by measuring changes in the surface plasmon coupling efficiency in the evanescent field. When changes occur in the refractive index of the sample material, the propagation of the evanescent wave and the angle of incidence producing resonance are affected. Therefore, by monitoring the angle of incidence at a given wavelength and identifying changes in the angle that causes resonance, corresponding changes in the refractive index and related properties of the material may be readily detected.

Nanoparticles of noble metals such as gold and silver are known to exhibit optical absorption and scattering properties in UV (approximately 10-380 nm)-visible (approximately 380-760 nm)-near IR region (Approximately (760-2,500 nm) termed as localized surface plasmon resonance (LSPR). The extinction band due to LSPR may be influenced by the size, shape and composition of nanoparticles and, most importantly by the surrounding environment. Refractive index changes taking place at the surface of the nanoparticles result in changes in absorbance and a red-shift in absorbance peak (λₘₐₓ). The LSPR properties of gold and silver nanoparticles may be utilized in liquid phase as well as in monolayers coated on glass/quartz substrates, for example, for detecting SARS-CoV-2 in a sample. One embodiment may include gold capped silica/polystyrene nanoparticle coated substrates. Other embodiments may include nanoparticles of rhodium, iridium, palladium, silver, osmium, iridium, platinum, or combinations thereof. The absorbance response of MNPs-based sensors may be further enhanced by coating MNPs on an efficient absorbance based sensor, such as a siloxane polymer. Sensitivity of optical fiber probes may be enhanced using the LSPR-based biosensor by using a fiber optic evanescent-wave sensing scheme. MNP coated on uncladded straight fiber probes may be used for chemical and biochemical sensing. In an alternative embodiment, the MNPs may be coated on a bent optical fiber. Various shapes of nanoparticles such as, round, cylindrical, rod shaped, etc. are within the scope of this disclosure. In some embodiments, the size of the nanoparticles (e.g., gold nanoparticles may be about 15 nm to about 60 nm).

It should be noted that for the label-free sensing approach described below, the metallic nanoparticles 120 or 220 may be gold nanoparticles because they exhibit LSPR. The particles may also be used for labeled sensing approaches to further increase sensitivity. It should be noted that LSPR eliminates the need for the use of polarized light. Similarly, use of LSPR eliminates the angle of incidence as a constraint for sensing.

In various embodiments the biosensor with a probe region 105 may include an uncladded portion of the optical fiber with an optional coating of metallic nanoparticles 120 or 220. As shown in FIG. 2B, bioreceptor molecules 201 may be immobilized on the probe region 105 (directly and/or over a gold nanoparticles substrate). Bioreceptor 201 may be, for example, any known antibody material and/or an engineered protein that can selectively bind SARS-CoV-2 and/or related biomolecules as the target substance. The antibody may be selected to biological properties of high binding affinity to specific target biomolecule. When the bioreceptor contacts a SARS CoV-2 sample, SARS CoV-2 related target biomolecules 202 are specifically and selectively attached to the bioreceptor, thereby capturing the target biomolecule at the bioreceptor. For example, during early stages of SARS CoV-2 infection, nucleocapsid protein (N-protein) is expressed in abundance in the host (about ing to about 1.5 ng/ml of a saliva sample), and the bioreceptor may be an antibody that selectively binds to the N-protein. Examples of such antibodies include, for example and without limitation, SARS-CoV/SARS-CoV-2 Nucleocapsid Protein (Rabbit) Antibody(Polyclonal) (Catalog number 200-401-A50, Rockland Immunochemicals, Inc.), SARS-CoV/SARS-CoV-2 Nucleocapsid Protein (Mouse) Antibody (Monoclonal) (Catalog number MAB8899, Abnova Corporation), SARS-CoV/SARS-CoV-2 Nucleocapsid Antibody, Mouse Mab (Catalog number 40143-MM05, Sino Biological, Inc.). Other antibodies that selectively bind to one or more of the S (Spike Glycoprotein), M (Membrane protein), E (Envelop small protein) and (HE) (hemagglutinin-esterase) proteins are also within the scope of this disclosure for use as a bioreceptor.

In other embodiments, the bioreceptor may be an antigen. Said antigen is specific for an antibody produced by the subject's body, for example in response to SARS-CoV-2 infection. Within the scope of the present invention, by “antigen” a substance, which is able to be recognized by antibodies of the subject's immune system, is meant. In particular, antigens used in the biosensor and diagnostic method of the present invention are peptides, natural proteins, fragments and epitopes thereof, or recombinant proteins containing at least one of such epitopes, recognized by specific antibodies, preferably for those serotypes of SARS-CoV-2 considered at high risk.

In some embodiments, a bioreceptor may be, for example, a nucleic acid, a poypeptide, a small organic molecule, cell, virus, bacteria, or the like.

Immobilization of one or more bioreceptor onto a probe region is performed so that it may not be washed away by rinsing procedures, and/or its binding to target biomolecules in the test sample is unimpeded by the probe surface. One or more specific bioreceptors may be attached to a probe surface by physical adsorption (i.e., without the use of chemical linkers) or by chemical binding (i.e., with the use of chemical linkers). Chemical binding can generate stronger attachment of specific binding substances on a probe surface and provide defined orientation and conformation of the surface-bound bioreceptor molecules. Examples of chemical binding include, for example, amine activation, thiol-PEG-NHS activation, aldehyde activation, and nickel activation. In some embodiments, the bioreceptor may be labeled to enhance the detection signal.

In various embodiments a “sandwich” or a “labelled” assay method 200A as disclosed with reference to FIG. 2A and FIG. 2C may be used with enhanced detection sensitivity. The nanoparticle-antibody conjugate 213 amplifies the detection signal because the target biomolecules 212 (e.g., N-protein) may potentially attach to double the amount of nanoparticles compared to the label free sensing modality shown in FIG. 2B. This also improves the sensitivity of the biosensor device such as a target biomolecular (and/or) load of as small as about 10³ to about 10⁶ particles/ml may be detected.

In some embodiments, bovine serum albumin (BSA) may further be added to the probe region 105 onto which the bioreceptor 101 is immobilized. Specifically, after bioreceptor 101 is immobilized on the probe region 105 (uncladded optical fiber with or without gold nanoparticle film,) BSA may be further added to an exposed area of the probe region 105 where bioreceptor 101 is not formed on the probe 105, to fill the exposed area of the probe 105. As such, BSA may serve to block the exposed areas of the probe region 105 and prevent other materials from non-specifically binding to the probe region 105. Milk protein or Mercaptohexanol and the like may be used instead of BSA in other embodiments.

In some embodiments, the probe region 105 may include more than one type of bioreceptor molecules. For example, the bioreceptor molecules may include a combination of N-protein binding antibodies and S-protein binding antibodies. Optionally, the probe region 105 may be divided into discrete sections (continuous and/or non-continuous), each associated with different bioreceptors.

In various embodiments the biosensor disclosed with reference to FIG. 1, the detector 106 may sense optical power loss in the light (or change in the absorbance/intensity count) propagating in the fiber-optic probe 104 due to the presence of a target biomolecule bound to the probe region 105. Any specific interaction between the bioreceptor and the biomolecule of interest leads to modulation in the effective refractive index (RI) and/or evanescent wave absorbance as a function of biomolecule concentration, and may be used for qualitatively and quantitatively calculating measured values of target biomolecules (e.g., concentration, presence/absence, etc.). The use of gold nanoparticles further amplifies the interaction signal that eventually leads to greater loss in the light and is measured usually as an increase in the absorbance value (or decrease in the intensity count). When a biological sample including a target biomolecule is supplied to the bioreceptor, the detector 106 may sense optical power loss generated either by labeling the bioreceptor with a specific label (e.g., gold nanoparticle) configured to generate optical power loss signals or by further adding a substance configured to generate optical power loss signals without labeling (i.e., label-free), and represents qualitatively and quantitatively measured values such as constituents, presence or absence and concentrations of the biological samples. The size, geometry, etc. of the label particle may be optimized to improve the sensitivity of the biosensor device. Various shapes of nanoparticles such as, round, cylindrical, rod shaped, etc. are within the scope of this disclosure. In some embodiments, the size of the nanoparticles (e.g., gold nanoparticles may be about 15 nm to about 60 nm).

In some embodiments, the concentration of a target biomolecule in a biological sample may be determined based on, for example, the radius of the optical fiber into which light is coupled, the numerical aperture at the sensing region of the fiber, the wavelength of light, the extinction coefficient of absorbing medium (GNP), and the concentration of absorbent molecules (i.e., bioreceptor) bound per unit circumferential surface area of the fiber, or the like. For example, the concentration may be proportional to the extinction coefficient, the wavelength of light at which the extinction is maximum (or a threshold), and/or the number of nanoparticles on the probe surface.

In various embodiments the detector 106 may further convert the optical signal to an electrical signal using any now or hereafter known techniques. The electrical signal may be transmitted to a processor of the biosensor device 100 and/or an external device for processing (i.e., analysis) such as determination of concentration, detecting presence or absence, and/or other properties of a target biomolecule. In one embodiment, it is possible to simplify the readout instrumentation by the application of a filter 108 so that only positive results over a determined threshold trigger detection.

In some embodiments, the biosensor device 100 may, optionally, include one or more of a processor 110 configured for, for example, analyzing the detected signal and generating an output; a controller 112 configured for, for example, controlling or programming the functionality of the biosensor device, including the light source, the detector and/or the optical fiber; a display 114 configured for, for example, displaying instructions, results, etc.; a power source 116 (e.g., via a USB connection); and/or other components (e.g., detector signal filters, etc.). One or more of the components may be included in the sensor device 100 and/or may be located externally and in communication with the sensor device 100 via an, optional, communications interface 118.

Furthermore, while not shown here, a sample holder may hold a biological sample from a subject and may be brought in contact with the probe region 105 of the biosensor device 100. For example, in an embodiment, the probe region 105 of the biosensor device 100 may be inserted into the sample holder such that it contacts the biological sample contained within. The sample holder may take a variety of configurations and in some embodiments the sample holder may be in the form of a cartridge, a cuvette, a test tube, a fluidic channel, a petri dish, a microtiter plate well, or the like. Optionally, an identifier (ID) detector may detect an identifier on the sample holder. The identifier detector may communicate may transmits the identifier to an external device (or a controller). Where desired, the external device and/or controller may identify a protocol to be run on the sample holder that may comprise instructions to the controller of the reader assembly to perform the protocol on the sample holder, including but not limited to a particular assay to be run and a detection method to be performed. Once the assay is performed on the sample holder, a signal indicative of a target biomolecule indicative of SARS-CoV-2 infection in the biological sample is generated and detected by the detector 106.

To ensure that a given sensor response (e.g. a light intensity) correlates with an accurate concentration of a target biomolecule of interest in a biological sample, the biosensor device may be calibrated before detecting the response using any now or hereafter known calibration protocols.

In some embodiments, detection of a SARS-CoV-2 infection in a biological sample may include illuminating the optical fiber 104 twice. The first measurement determines the absorption spectra with one or more specific bioreceptors immobilized on the probe region surface. The second measurement determines the absorption spectra after one or more target molecules are applied to a probe region. The difference in between these two measurements is a measurement of the amount of target biomolecules that have specifically bound to bioreceptor in the probe region. This may account for nonuniformities the probe region as well as varying concentrations or molecular weights of immobilized bioreceptors.

In various embodiments the method of detecting target biomolecules in a biological sample indicative of SARS CoV-2 in different concentrations in a bodily fluid from a subject. The method may include providing a biosensor device comprising an optical fiber sensor having a functionalized probe region; allowing a biological sample of bodily fluid to react with a bioreactor of the functionalized probe region; causing light to traverse the optical fiber; and detecting signals that are indicative of the presence or absence of the target biomolecules, wherein the detected signals may include an optical power of the light propagating in the fiber-optic probe and where presence of the target biomolecule causes optical power loss (or change in the absorbance/intensity count). The methods may be used diagnostically to, for example, assess if a subject has been infected with SARS-CoV-2 or monitor the development or progression of a SARS-CoV-2 infection as part of a clinical testing procedure to, e.g., determine the efficacy of a given treatment regimen.

In various embodiments the (potentially) infected subjects may be human subjects, but also animals that are suspected as carriers of SARS-CoV-2 might be tested for the presence of SARS-CoV-2. The biological sample may first be manipulated to make it more suitable for the method of detection. Manipulation may include treating the biological sample suspected to contain and/or containing SARS-CoV-2 in such a way that the SARS-CoV-2 will disintegrate into antigenic components such as proteins, (poly)peptides or other antigenic fragments. Preferably, the bioreceptors are contacted with the biological sample under conditions which allow the formation of an immunological complex between the bioreceptor molecules and SARS-CoV-2 or antigenic components thereof that may be present in the sample. The formation of an immunological complex, if any, indicating the presence of SARS-CoV-2 in the sample, is then detected and measured, as discussed above.

In various embodiments the wide range of pathogen (i.e., SARS CoV-2) concentrations in a sample from a subject may be detected either directly or indirectly are discussed. The amount of pathogen present in a test sample may be expressed in any of a number of ways well known in the art. By way of non-limiting examples, the number of pathogens or target biomolecules associated with pathogens may be expressed as viral burden, infectious units (IU), and/or infectious units per million cells or milliliter (IUPM), number of particles/ml, or the like. In one example, it is envisioned that pathogens or target biomolecules associated with pathogens may be detected in a test sample at a concentration of as low as 10 fg to about 50 ng/ml.

In various embodiments the present disclosure also provides a method of monitoring more than one pharmacological parameter useful for assessing efficacy and/or toxicity of an anti-SARS COV-2 therapeutic agent. The method may include subjecting a biological sample from a subject administered with the anti-SARS COV-2 therapeutic agent to a biosensor device of this disclosure for monitoring said more than one pharmacological parameter to yield detectable signals indicative of the values of the more than one pharmacological parameter from said sample; and detecting said detectable signal generated from said biological sample. Where desired, the method further involves repeating the steps at a time interval. For the purposes of this disclosure, a “therapeutic agent” is intended to include any substances that have therapeutic utility and/or potential. Such substances include but are not limited to biological or chemical compounds such as a simple or complex organic or inorganic molecules, peptides, proteins (e.g. antibodies) or a polynucleotides (e.g. anti-sense). A vast array of compounds may be synthesized, for example polymers, such as polypeptides and polynucleotides, and synthetic organic compounds based on various core structures, and these are also included in the term “therapeutic agent”. In addition, various natural sources can provide compounds for screening, such as plant or animal extracts, and the like. It should be understood, although not always explicitly stated that the agent is used alone or in combination with another agent, having the same or different biological activity as the agents identified by the inventive screen. The agents and methods also are intended to be combined with other therapies.

In some embodiments the SARS-Cov-2 biosensors using fiber optic probes in accordance with the present disclosure have the advantages of portability, ease of use to enable point-of-care applications, low cost, quantitative testing, fast delivery of results, and improved sensitivity, selectivity and reliability. While exemplary embodiments have been presented in the foregoing detailed description of the embodiments, it should be appreciated that a vast number of variations exist.

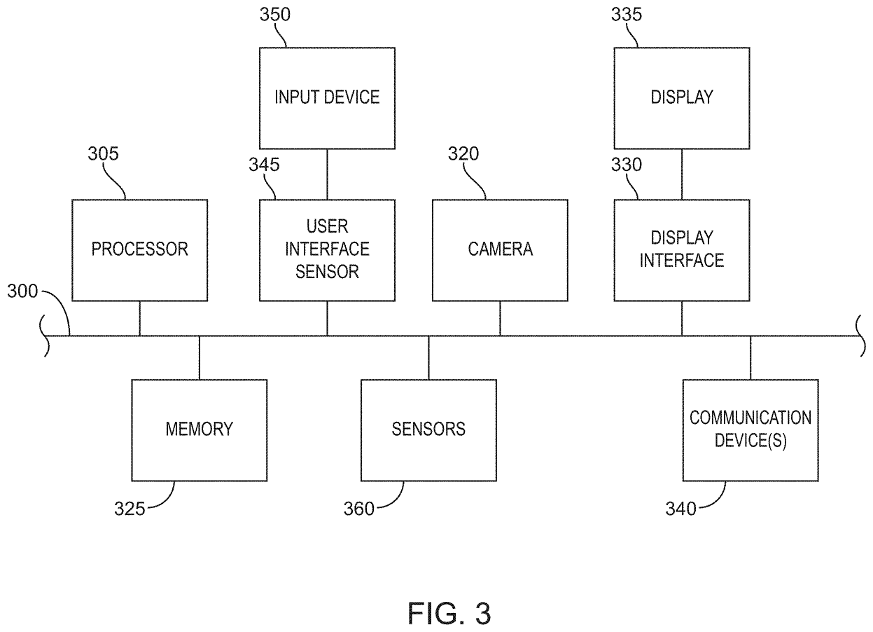

In various embodiments the FIG. 3 depicts an example of internal hardware that may be included in any of the electronic components of the biosensor device, such as the controller (or components of the controller), processor, detector, etc. described above. An electrical bus 300 serves as an information highway interconnecting the other illustrated components of the hardware. Processor 305 is a central processing device of the system, configured to perform calculations and logic operations required to execute programming instructions. As used in this document and in the claims, the terms “processor” and “processing device” may refer to a single processor or any number of processors in a set of processors that collectively perform a set of operations, such as a central processing unit (CPU), a graphics processing unit (GPU), a remote server, or a combination of these. Read only memory (ROM), random access memory (RAM), flash memory, hard drives and other devices configured to store electronic data constitute examples of memory devices 325. A memory device may include a single device or a collection of devices across which data and/or instructions are stored. Various embodiments of the invention may include a computer-readable medium containing programming instructions that are configured to cause one or more processors, print devices and/or scanning devices to perform the functions described in the context of the previous figures.

In various embodiments an optional display interface 330 may permit information from the bus 300 to be displayed on a display device 335 in visual, graphic or alphanumeric format. An audio interface and audio output (such as a speaker) also may be provided. Communication with external devices may occur using various communication devices 340 such as a wireless antenna, an RFID tag and/or short-range or near-field communication transceiver, each of which may optionally communicatively connect with other components of the device via one or more communication system. The communication device(s) 340 may be configured to be communicatively connected to a communications network, such as the Internet, a local area network or a cellular telephone data network.

In various embodiments the hardware may also include a user interface sensor 345 that allows for receipt of data from input devices 350 such as a keyboard, a mouse, a joystick, a touchscreen, a touch pad, a remote control, a pointing device and/or microphone. Digital image frames also may be received from a camera 320 that can capture video and/or still images. The system also may receive data from other sensors 360 such as a barcode sensor, an positional sensor, or the like.

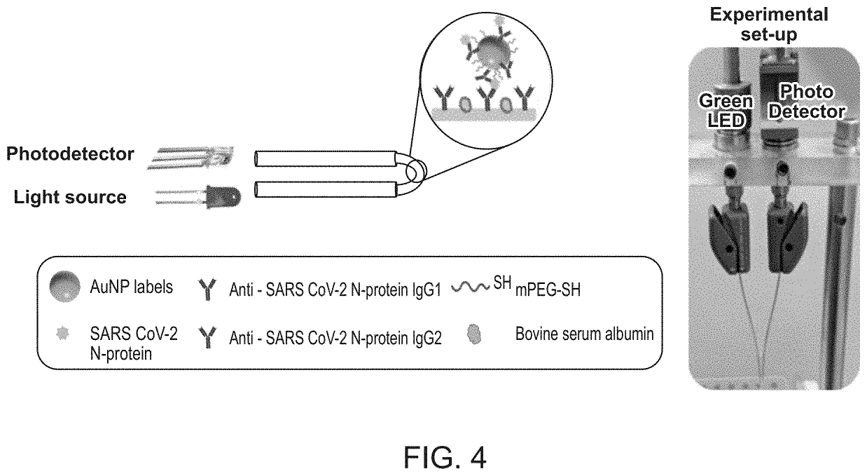

In various embodiments the plasmonic fiber optic absorbance biosensor (P-FAB) strategy for the detection of SARS-CoV-2 N-protein and the LED-PD based experimental set-up used for the optical absorbance measurements are shown in FIG. 4.

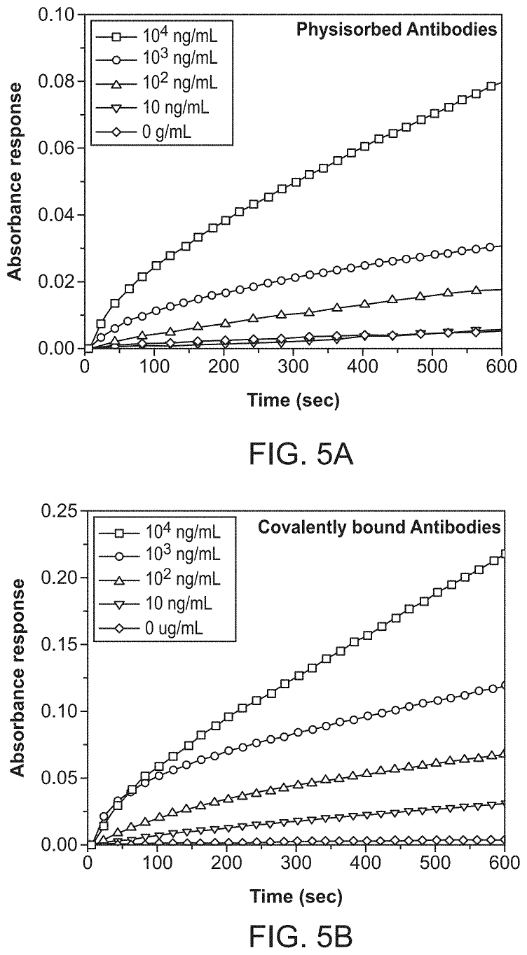

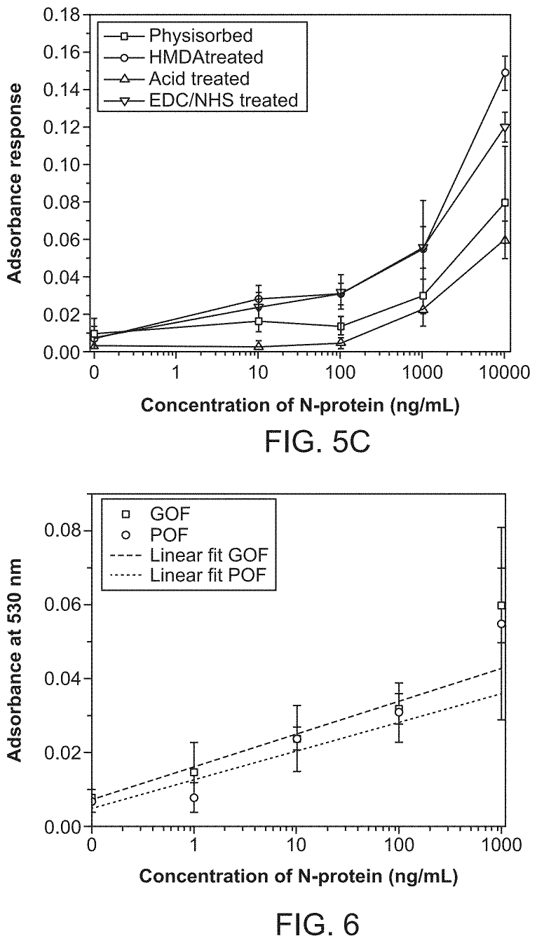

In various embodiments the temporal absorbance response from U-bent POF probes coated with capture antibodies are discussed in reference to FIGS. 5A and 5B by means of physisorption, (FIG. 5A) and covalent binding (FIG. 5B). FIG. 5(C) shows dose-response curve obtained from POF sensor probes with capture antibody (Anti-SARS N-protein IgG) immobilized by means of physisorption, Acid-treatment, HMDA, and EDC/NHS based covalent binding methodology using a green LED-photodetector (PM100, S150c) setup due to binding of immunocomplex with AuNP labels (40 nm, 10×) conjugated with anti-SARS CoV2 -N-protein IgG2.

In various embodiments the absorbance response obtained from POF and GOF sensor probes chemisorbed with anti-SARS-CoV-2 -N-protein IgG1 with the help of a green LED-photodetector (PM100, S150c) setup due to binding of immunocomplex with AuNP labels (40 nm, 10×) conjugated with anti-SARS-COV-2-N-protein IgG2 resuspended in PBS and BSA-based PBS buffer are discussed in reference to FIG. 6.

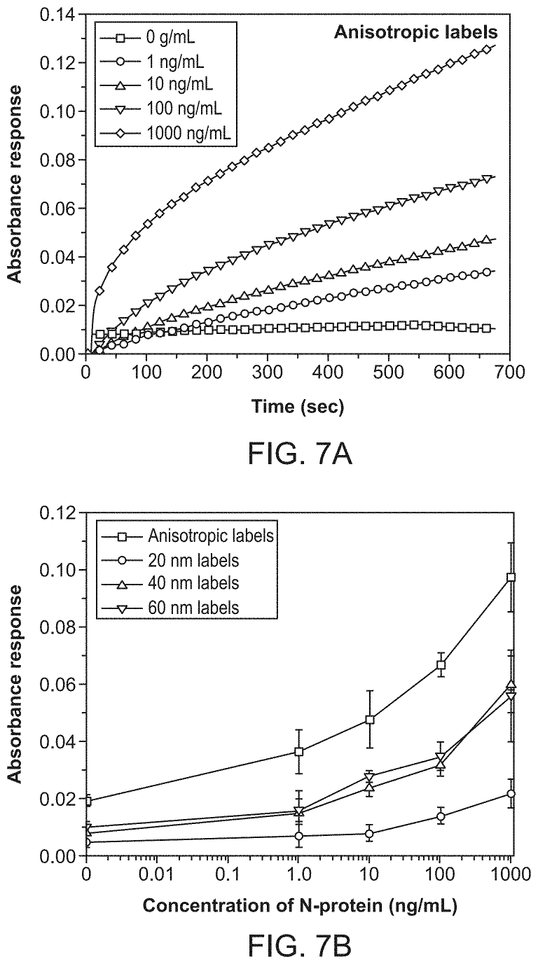

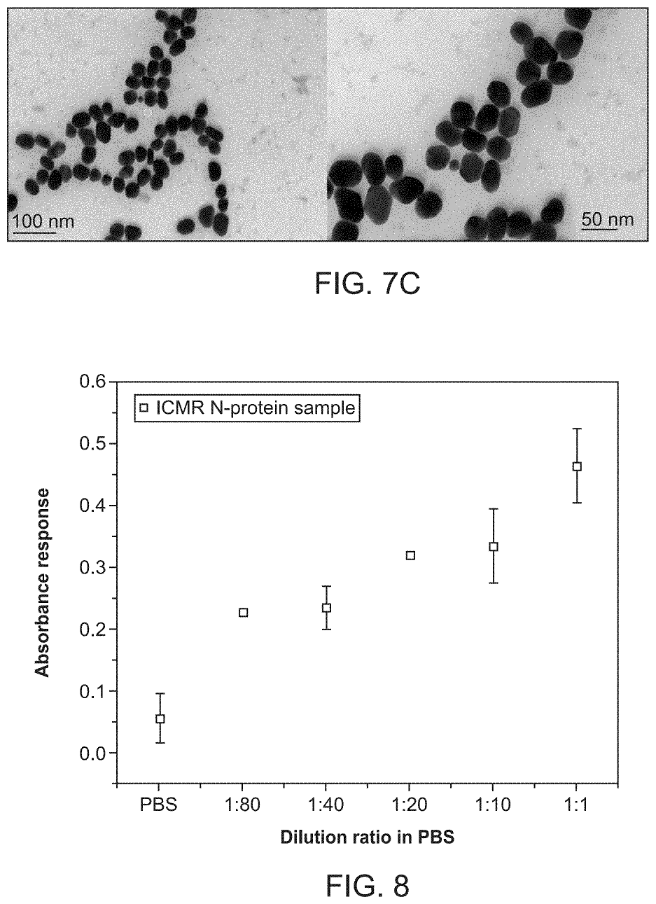

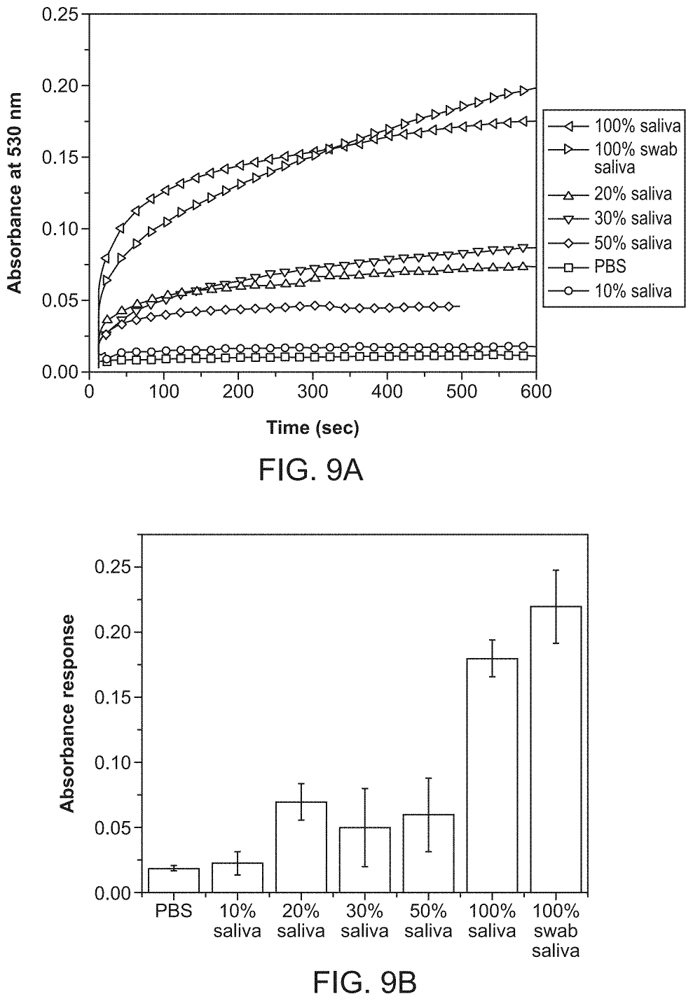

In various embodiments the temporal absorbance response curves for prepared anisotropic elliptical AuNP are shown in FIG. 7A and In FIG. 7B dose-response curves obtained from GOF sensor probes chemisorbed with anti-SARS-CoV-2-N-protein IgG1 with the help of the optical experimental setup due to binding of immune complex with AuNP labels varying sizes and 10× concentration conjugated with anti-SARS CoV-2-N-protein IgG2 resuspended in PBS. FIG.7C shows TEM images of prepared anisotropic or elliptically shaped AuNPs. In various embodiments the dose response curve of sensor probes for various dilutions of SARS-CoV-2 N-protein are described in reference to FIG. 8 that is the dose response curve obtained using covalently immobilized capture antibody functionalized sensor probes for various dilutions of SARS-CoV-2 N-protein standard reference sample obtained from ICMR (n=2). In various embodiments the absorbance response of sensor probes using P-FAB strategy is analyzed with different dilutions of saliva are shown in FIG. 9.

The above-disclosed features and functions, as well as alternatives, may be combined into many other different systems or applications. Various presently unforeseen or unanticipated alternatives, modifications, variations or improvements may be made by those skilled in the art, each of which is also intended to be encompassed by the disclosed embodiments. While the invention has been disclosed with reference to certain embodiments, it will be understood by those skilled in the art that various changes may be made and equivalents may be substituted without departing from the scope of the invention. In addition, many modifications may be made to adapt to a particular situation or material the teachings of the invention without departing from its scope, which should be as delineated in the claims to follow.

### EXAMPLES

### Example 1: Fabrication of U-Bent Fiber Optic Probes

1.1.1 Plastic optical fiber (POF) probes: The plastic optical fiber consists of polymethylmethacrylate (PMMA) core and fluorinated polymer cladding with refractive indices of 1.49 and 1.41, respectively. The POF of 0.5 mm core diameter was bent into U-shape with a bend ratio (bend diameter/core diameter) of 3 using glass capillary based thermal treatment method as reported elsewhere (Gowri and Sai, 2016). Briefly, the 22 cm long POF is bent to bring the two ends close to each other and pushed into a glass capillary having an optimal inner diameter. Subsequently, the glass capillary loaded with POF was placed in a hot air oven and heated at 100° C. for 10 min in order to obtain a permanent deformation into a U-shape. U-bend region of the probe was dipped in ethyl acetate for 2 min to remove the cladding over a length of 5 mm. Then, the U-bent sensing portion was wiped with a lint-free tissue to remove the flakes over the bent portion and cleaned with DI water.

1.1.2 Fused silica optical fiber (GOF) probes: The glass optical fibers (GOFs) consist of a fused silica core and silica clad, and a polymer buffer layer. U-bent GOF probes with optimal bend diameter were made using a customized CO₂ laser bending machine. Briefly, a 20 cm long GOF with a 200 μm core diameter was subjected to the bending process with the help of a built-in-house fiber bending machine, which is equipped with a CO₂ laser and motors capable of performing buffer ablation followed by bending of a straight fiber with a desired bend diameter. Once after the bending process, the U-bent fiber probes were sonicated and wiped with acetone to remove the black char and debris of the polymer buffer layer. The U-bent region of the fiber probes was dipped into 40% HF solution for 5 minutes to remove the fused silica clad. (Note: The 5 minutes of etching time was optimized by visual examination of the diameter of fiber probes through a microscope every few min of etching. The fiber diameter was measured after every minute for a reduction from 220 μm to less than 200 μm to ensure complete etching of fluorinated silica clad layer. Once after HF etching, the probes are washed with DI water and followed by acetone sonication for 2 to 5 mins.

1.2 Functionalization of U-bent sensor probes: The U-bent sensor probes were treated with suitable chemical agents to generate functional surface for the immobilization of capture antibody molecules (anti-SARS CoV-2 N-protein immunoglobulin G (IgG1), (procured from Meridian Life Science, Inc., USA)).

1.2.1 POF sensor probes: (i) Acid treatment: In this process, the U-bent POF sensor probes were treated with 1 M H₂SO₄ for 1 hr followed by incubating in methanol:HCl (1:1) for 1 hr at room temperature (RT) to generate hydroxyl (—OH) groups on the surface. Later, the acid treated sensor probes were directly utilized for capture antibody immobilization.

(ii) Ethyl(dimethylaminopropyl) carbodiimide/N-hydroxysuccinimide (EDC/NHS): The U-bent decladded region of the fiber probe was incubated in methanol:HCl (1:1) for 1 hr at room temperature (RT) to generate hydroxyl groups on the sensor probe surface. Then, the hydroxylated sensor probes were dipped in EDC (100 mM), and NHS (200 mM) (prepared in 0.1 M MES buffer in 0.9% NaCl, pH 6) over a duration of 1 hr, followed by heat treatment at 60° C. for 20 mins. Then, the sensor probes were washed with MES buffer.

(iii) Hexamethylenediamine The U-bent decladded region of the fiber probe was incubated in 1 M H₂SO₄ for 5 mins at RT to generate hydroxyl groups on the sensor probe surface. Then, the amination was carried out by incubating the U-bent sensor probe in 10% v/v HMDA (in 100 mM borate buffer) for 2 h. Then, the fiber probes were washed with borate buffer and subsequently condensed at 60° C. for 15 mins. Further, the aminated sensor probes were incubated in 2.5% glutaraldehyde for 30 mins at RT to generate aldehyde rich surface.

1.2.2 GOF sensor probes: The U-bent silica fiber probes were cleaned by sonication in acetone (15 min, 1000 Watt, 28 kHz). The cleaned U-bent sensing region of the fiber probes was further cleaned with piranha solution (20 min, 60° C.) to oxidize and remove any organic contamination as well as generate hydroxyl groups on the sensor surface. Thereafter, the fiber probes were washed with deionized water and dehydrated for 1 h at 115° C. in order to remove physisorbed water. For amino-silanization the fiber probes were dipped in a 1% solution of APTMS in a 5:2 (v/v) mixture of ethanol and acetic acid (5 min). Finally, the fiber probes were washed thrice in ethanol, sonicated (15 min) and dried (100° C., 1 h). Then, the silanized sensor probes were dipped into 1% glutaraldehyde (500 μL, 30 min, RT) to establish aldehyde groups.

1.3 Capture antibody immobilization on U-bent sensor probes: The functionalized POF and GOF sensor probes were immobilized with anti-SARS CoV-2 N-protein IgG1, referred as capture antibody. Briefly, the functionalized U-bent sensor probes were dipped in 50 μL of 50 μg/mL of capture antibody solution overnight at 4° C. Later, the antibody immobilized sensor probes were washed with PBS and dipped in 50 μL of 5 mg/mL of bovine serum albumin (BSA) solution over a duration of 15-20 mins in order to passivate/block the free functional (—OH and —CHO) groups on the sensor probe surface.

1.4 Synthesis of gold nanoparticles (AuNPs) and preparation of AuNP label conjugates: The gold nanoparticles (AuNP) were synthesized by citrate mediated reduction of HAuCl4 (Turkevich in 1951). Briefly, 1 mL of 12.7 mM gold chloride salt was added to 38 mL of DI water and heated. An aqueous solution of trisodium citrate dihydrate (0.349 mM, 1 mL) was added to the boiling solution of gold chloride salt, while citrate to gold molar ratio was maintained as 1.1. The heating was continued until the solution color turns pale pink indicating the formation of AuNP. Thereafter, the solution was allowed to cool to room temperature (RT) and stored at 4° C. The colloidal solution of AuNP was characterized using UV-vis spectroscopy and transmission electron microscopy (TEM) for its optical absorption characteristics and their size and shape distribution respectively. UV-vis spectroscopic analysis shows a strong absorption peak at 530 nm. TEM analysis reveals the presence of anisotropic AuNPs of average size of 40 nm.

The plasmonic AuNP labels were prepared by utilizing the affinity of amine and thiol groups on the detection antibodies towards the gold nanoparticles. Commercial AuNPs of size 20, 40 and 60 nm (BBI solutions, UK) were also used as labels. AuNP solution (1 mL, OD ˜1, pH 8.5) was taken and 100 μl of 25 μg/ml of anti-SARS CoV-2 N-protein IgG 2 solution (procured from Meridian Life Science, Inc., USA) was added and incubated for 15 mins at RT. Soon after, 80 μL of 320 μM SH-PEG was added and incubated for 15 mins. Then, the reaction mixture was centrifuged at varying RPM depending on the size of the AuNP (20 nm was centrifuged at 11000 rpm and 40 nm, 60 nm and anisotropic AuNP was centrifuged at 8500 rpm) for 20 mins at 4° C. to remove any unbound and loosely bound antibodies. The supernatant was discarded, and the AuNP labels were resuspended using 100 μl of resuspension buffer containing BSA, sodium salts, sucrose, trehalose, Tween-20, and sodium azide (VoxturBio Pvt., Ltd.,) to obtain 10× concentrations of AuNP labels.

1.5 Optical absorbance measurements: The optical set-up for P-FAB involves a pair of a simple LED and a photodetector. A capture antibody immobilized sensor probe was coupled between a green LED light source (525 nm wavelength, built-in-house) and the photodetector (S150C, Thorlabs Inc., USA) using bare fiber adaptors. The intensity of the light propagating through (at a particular wavelength of choice, 530 nm) the sensor probe was monitored in real-time and recorded in a PC through PM 100 console (Throlabs Inc., USA). The drop in optical intensity occurs due to the formation of the immunocomplex on the sensor probe surface. Later, an absorbance response was derived from the temporal sensor response by taking the logarithmic ratio of initial intensity and final intensity, mainly to appreciate even small changes in the intensity.

1.6 Plasmonic fiber optic absorbance biosensor (P-FAB) strategy: The P-FAB strategy involves realization of a plasmonic sandwich immunoassay using AuNP labels on a U-bent fiber optic sensor probe as depicted in FIG. 1. Firstly, the sample solution containing the analyte molecules (SARS-CoV-2 N-protein, procured from GenScript, USA) was homogeneously mixed with plasmonic AuNP label reagent, each of 25 μL volume. The sample-reagent mixture was kept undisturbed for 10 mins at RT to facilitate the capturing of analyte molecules by the plasmonic labels leading to formation of AuNP-IgG2-SARS CoV-2 N-protein complex. After the incubation time, the sensor probe connected to the optical set-up is dipped into the sample-reagent mixture containing the AuNP-SARS CoV-2 N-protein complex. A sandwich immunocomplex (AuNP-IgG2-SARS CoV-2 N-protein-IgG complex) formation is achieved on the fiber probe surface. The drop in optical intensity as a result of the formation of the immunocomplex to the sensor probe was monitored in real-time and recorded using the optical set-up.

### Example 2: Realization of P-FAB for the Detection of SARS-CoV-2 N-Protein

Given the recent attention gained by plastic optical fibers as an alternative to the glass optical fibers, this study investigated the potential of U-bent POF probes as a possible candidate in addition to the conventional glass optical fibers with an established surface modification technique.

2.1.1 U-bent POF sensor probes: In order to realize U-bent POF probe based P-FAB for N-protein detection, optimum conditions for antibody immobilization on POF probe surface were investigated. Capture antibody immobilized U-bent POF probes obtained in four different methods including (i) simple physisorption of antibodies without any surface pre-treatment, (ii) antibodies directly immobilized over an H₂SO₄ acid-treated POF probes, (iii) EDC/NHS treated probes with hydroxyl/carbonyl activated POF surface and (iv) covalent immobilization over HMDA/glutaraldehyde treated POF probes. Sandwich assay was realized for each of the above-mentioned POF surface modification methods. The P-FAB response for different concentrations (0, 10 to 10,000 ng/mL) of N-protein dissolved in PBS buffer was obtained as described in the Sec. 2.6. FIG. 5A and FIG. 5B show the temporal response from the U-bent POF probes obtained by physisorption and HMDA based covalent immobilization of antibodies. The absorbance response for each N-protein concentration was recorded for 10 min. Dose response curves for all the four surface pre-treatment methods were compared in FIG. 5C. For the surface functionalization strategies involving the physisorption of antibodies with or without acid treatment, the sensor response for 0 ng/mL and 10 ng/mL was indistinguishable. The dose response curves show a considerably lower response for higher analyte concentrations with a high standard deviation in comparison to the covalent immobilization strategies involving HMDA/glutaraldehyde or EDC/NHS based antibody binding. The relatively poor performance in case of physisorption based antibody immobilization may be attributed to either the steric hindrance effects or a lower surface coverage of antibodies, which requires a detailed a detailed investigation. The sensitivity of the POF-based sensor probes considering the sensor response between 0 to 1000 ng/mL, using HMDA and EDC/NHS-based strategies was found to be 0.005 A530 nm/log ng/mL and 0.006 A530 nm/log ng/mL with R-square values 0.97 and 0.84 respectively. The R-square values signify the non-linearity in the sensor response. The detection limit calculated using the formula 3σ/S gives rise to 1.8 ng/mL and 3 ng/mL for HMDA and EDC/NHS-based strategies respectively. Subsequently, HMDA based covalent antibody immobilization was chosen for POF probes.

### Example 3: Sensitivity of U-Bent GOF and POF Sensor Probes

The sensor response of HMDA functionalized POF and GOF sensor probes with covalent immobilization of antibodies were compared in order to identify the sensor probe with higher sensitivity to realize the detection of SARS-CoV-2-N-protein. The sensitivity of the POF and GOF sensor probes within the dynamic range of 0 ng/mL to 1000 ng/mL were found to be 0.007 A530 nm/log ng/mL and 0.008 A530 nm/log ng/mL with an R-square value of 0.80 and 0.95, respectively (FIG. 6). The detection limits were calculated using 3σ/S give rise to 1.2 ng/mL and 0.75 ng/mL. The GOF sensor response was found to be repeatable and with lesser standard deviation. Hence, GOF was identified as the optimum probe for P-FAB strategy to realize SARS-CoV-2-N-protein detection. The improved sensitivity of the GOF sensor probes is in agreement with the previous publication for the detection of M.TB LAM in urinary samples (Divagar et al., 2020). However, the sensitivities values are much different in comparison to previously reported values, which could be attributed to the change in the sensor probe from polymer cladded GOF sensor probes to fused silica cladded GOF sensor probes, decladding process, analyte being detected, and its molecular weight and the binding affinities of the antibodies used.

2.2 Optimum size of AuNP labels: The high sensitivity of the P-FAB-based sensing strategy originates from the unique ability of the U-bent optical fiber probes to detect the plasmonic AuNP labels with a large optical extinction coefficient binding to the bend region. The influence of AuNP label size on the P-FAB response is multi-faceted. On one hand, the optical extinction property of these AuNP labels, which enables the ultra-low analyte detection limits, is highly dependent on their size (Divagar et al., 2020). On the other hand, the number of detector antibodies bound to the AuNP labels and the accessibility of their binding sites for the analyte molecules is also influenced by the AuNP size. Moreover, the concentration of the AuNP conjugated with detector antibodies is another critical factor that determines the availability of a sufficient number of AuNP bioconjugates for efficient capture of analytes and the diffusion of the immunocomplex in the solution phase towards the probe surface. In addition, the optimum AuNP label size and concentration are also be governed by the molecular weight of the analyte of interest as well as its concentration dynamic range of interest. Hence, AuNP labels of four different distributions including highly uniform sizes around 20, 40 and 60 nm (procured from BBI solutions, UK) as well as asymmetric AuNP prepared-in-house were investigated. The P-FAB response to various concentrations of N-protein for each of the four AuNP distributions was obtained. The representative temporal absorbance response curves for anisotropic elliptical AuNP prepared-in-house are shown in FIG. 7A. FIG. 7B shows the dose response curves obtained for each of the AuNP size distribution.

It may be noted that AuNP 20 nm labels gave rise to a relatively poor sensitivity to analyte concentrations, in comparison to the 40 and 60 nm AuNP of highly uniform morphology. However, the AuNP labels with asymmetric elliptical shape with an average size of 40 nm resulted in a much higher sensitivity in comparison to the highly controlled size distributions in the range of 40 and 60 nm. The sensitivity of the GOF sensor probes for the detection of SARS-CoV-2-N-protein using the anisotropic labels within the dynamic range of 1 to 1000 ng/mL was found to be 0.016 A530 nm/log ng/mL with an R-square value of 0.95. The detection limit calculated using 3σ/S gives rise to 0.37 ng/mL. The improved sensor response and sensitivity could be attributed to the larger size distribution and the irregular shape leading to improved extinction co-efficient as well as improve accessibility of paratopes of the antibodies bound to the AuNP labels. All the subsequent studies were carried out with the lab-prepared AuNP.

2.3 Validation of the P-FAB using standard reference N-protein samples: P-FAB was realized using a standard reference sample for N-protein as simulated analyte, provided by Indian council for medical research (ICMR) for validation of antigen based diagnostic kits (Courtesy: Voxtur Bio Ltd, Mumbai, India). Under the optimum conditions using U-bent GOF probes and lab-prepared anisotropic AuNP labels, the P-FAB response for various dilutions of N-protein reference sample down to 80× (in PBS) was obtained as shown in FIG. 8. In comparison to PBS solution without N-protein, a distinguishable response was observed for N-protein dilutions as low as 80×. While the absolute concentration of N-protein in the standard reference is not known, the conventional AuNP label based lateral flow assay antigen test kits were sensitive to a dilution down to 1:40 (Courtesy: VoxturBio Ltd). On the other hand, the P-FAB response demonstrates its ability to detect analyte concentrations below 1:80 dilution. The sensitivity of the GOF sensor probes for the detection of SARS-COV-2-N-protein within the dynamic range of 1:80 to 1:1 was found to be 0.15 A530 nm/log SARS-CoV-2 N-protein percentage (%) with an R-square value of 0.90. The detection limit calculated using 3σ/S gives rise to 0.8%, which corresponds to 1:125 dilution.

2.4 Clinical sample analysis: Two samples that are preferred for the analysis includes saliva and oropharyngeal swabs. In both the cases the samples are to be extracted using an extraction buffer in order to get the N-protein for detection. In case of saliva mucus and the other constituents could potentially interfere with the sensor response leading to a potentially higher non-specific binding (NSB). Hence, artificial saliva at various dilutions (prepared in PBST buffer, PBS containing 1% TritonX-100, pH 7.4) without N-protein were investigated to quantify the non-specific binding of AuNP labels and choose an appropriate dilution for reduced NSB. In addition, a cotton swab based filtration of saliva was implemented to understand the influence of the mucus substance over the sensor response. A sandwich assay with saliva as sample without containing any N-protein carried out as described in Sec. 2.6.FIG. 9A shows the temporal absorbance response due to NSB from PBS alone and the sample containing only 10%, 20%, 30%, 50% or 100% saliva as well as the cotton swab based mucus-filtered saliva (neat and undiluted). FIG. 9B compares the absorbance response from the sensors subjected to these various dilutions of saliva, obtained by the end of 10 min of incubation. The NSB for 10% saliva samples was the lowest in comparison to the samples containing a higher percentage of saliva as much as that of PBS, demonstrating minimal influence of saliva constituents on the NSB of AuNP labels. On the other hand, 100% mucus-filtered saliva has shown a slightly higher NSB in comparison to that of 100% saliva, indicating a potential interference from the cotton swab leading to an increase in NSB. These results suggest the use of 10× dilution of saliva samples for the assay reduces the presence of interfering molecules in the saliva and minimize the NSB of AuNP labels.

We claim:

1. A biosensor device 100 for detecting Severe Acute Respiratory Syndrome Coronavirus 2 (SARS-CoV-2) or fragment thereof in a biological sample, the biosensor device comprising:
an optical fiber 104 comprising:
at least one curved portion 104a, and
a probe region 105, the probe region comprising a plurality of immobilized bioreceptor molecules 201 configured to bind to target biomolecules associated with SARS CoV-2 infection in the subject;
a light source 102 located proximal to one end of the optical fiber 104; and
a detector 106 located proximal to another end of the optical fiber 104, wherein the detector is configured to sense a change in an optical property of light that traverses through the optical fiber when the probe region 105 is contacted with a biological sample including the target biomolecules.

2. The biosensor device as claimed in claim 1, wherein the probe region 105 comprises a coating 120 of gold or silver nanoparticles.

3. The biosensor device as claimed in claim 1, wherein the plurality of bioreceptor molecules comprise an antibody configured to bind to an antigen of the SARS-CoV-2.

4. The biosensor device as claimed in claim 3, wherein the antigen is one or more of N (nucleocapsid (N) glycoprotein) according to SEQ. ID. No. 1, S (Spike Glycoprotein) according to SEQ. ID. No. 2, M (Membrane protein) according to SEQ. ID. No. 3, or E (Envelop small protein) according to SEQ. ID. No. 4, of the SARS-CoV-2.

5. The biosensor device as claimed in claim 1, wherein the biological sample comprises saliva, nasopharyngeal or oropharyngeal swab collected from a subject.

6. The biosensor device as claimed in claim 4, wherein the bioreceptor molecules are anti-SARS CoV-2 polyclonal or monoclonal antibody against the antigen.

7. The device of claim 1, wherein the optical fiber is made of a transparent material selected from silica, quartz, polymethyl methacrylate, polystyrene, ceramic glass, or chalcogenide glass.

8. The device of claim 2, wherein the nanoparticles 120 or 220 are spherical or elliptical gold nanoparticles of size 15-60 nm.

9. A labelled assay method for detecting Severe Acute Respiratory Syndrome Coronavirus 2 (SARS-CoV-2) in a sample, the method comprising:
providing (401) an optical probe biosensor device having a U-bent probe region;
immobilizing (403) a bioreceptor configured to bind to target biomolecules associated with SARS CoV-2 to the probe region;
mixing (405) the biological sample with gold nanoparticle labels conjugated with a biorecognition molecule specific to a SARS-CoV-2 antigen and incubating it to allow formation of an AuNP-antibody-antigen complex;
exposing (407) the probe region to the sample-label mixture to allow binding of the target biomolecules and formation of a sandwich immunocomplex with gold nanoparticle labels;
passing light through the optical fiber and detecting (409) a change in intensity of the light passing through the optical probe biosensor as a function of the amount of target biomolecules associated with SARS CoV-2 forming the immunocomplex.

10. The method as claimed in claim 9, comprising functionalizing (402) the U-bent sensor probe surface with —OH or —CHO groups prior to immobilization of the bioreceptor.

11. The method as claimed in claim 9, wherein the bioreceptor molecule or the biorecognition molecule comprise an antibody configured to bind to an antigen of the SARS-CoV-2, selected from one or more of N (nucleocapsid (N) glycoprotein) according to SEQ. ID. No. 1, S (Spike Glycoprotein), according to SEQ. ID. No. 2, M (Membrane protein) according to SEQ. ID. No. 3, E (Envelop small protein) according to SEQ. ID. No. 4, or (HE) (hemagglutinin-esterase) protein of the SARS-CoV-2.

12. A label-free assay method for detecting Severe Acute Respiratory Syndrome Coronavirus 2 (SARS-CoV-2) in a sample, the method comprising:
providing (501) an optical probe biosensor device having a U-bent probe region;
providing (503) a coating of gold nanoparticles on the U-bent probe region;
immobilizing (505) a bioreceptor configured to bind to target biomolecules associated with SARS CoV-2 to the nanoparticle-coated probe region ;
exposing (507) a biological sample to the probe region to cause the target biomolecules to bind to the bioreceptor and form an immunocomplex;
passing light through the optical fiber and detecting (509) a change in intensity of the light passing through the optical probe biosensor as a function of the amount of target biomolecules associated with SARS CoV-2 forming the immunocomplex.

13. The method as claimed in claim 12, comprising functionalizing (502) the U-bent sensor probe surface with —SH or —NH2 groups prior to coating with gold nanoparticles and immobilizing the bioreceptors.

14. The method as claimed in claim 12, wherein the bioreceptor molecule comprises an antibody configured to bind to an antigen of the SARS-CoV-2, selected from one or more of N (nucleocapsid (N) glycoprotein) according to SEQ. ID. No. 1, S (Spike Glycoprotein), according to SEQ. ID. No. 2, M (Membrane protein) according to SEQ. ID. No. 3, or E (Envelop small protein) according to SEQ. ID. No. 4, of the SARS-CoV-2.
